(19) United States
(12) Patent Application Publication
(10) Pub. No.: US 2026/0013734 A1
(43) Pub. Date: Jan. 15, 2026

(54) ARTIFICIAL INTELLIGENCE WEARABLE KIT OF MONITORING RESPIRATION AND HEART CONDITION AND SYSTEM THEREFOR

(71) Applicant: Kuo-Yuan Chang, New Taipei City (TW)

(72) Inventor: Kuo-Yuan Chang, New Taipei City (TW)

(21) Appl. No.: 18/821,759

(22) Filed: Aug. 30, 2024

(30) Foreign Application Priority Data
Jul. 9, 2024 (TW) 113125717

(51) Int. Cl. A61B 5/0205; A61B 5/00; A61B 5/024; A61B 5/08; A61B 5/1455; G16H 50/30

(52) U.S. Cl. CPC A61B 5/0205; A61B 5/681; A61B 5/743; G16H 50/30; A61B 5/02427; A61B 5/0816; A61B 5/14552; A61B 2560/045; A61B 2560/0462; A61B 2562/0219

(57) ABSTRACT

A wearable respiration-heart monitoring kit includes a detection finger band and a wearable heart rate relay watch. The detection finger band includes a light-emitting unit for emitting a detection light beam and a responsive light receiving unit for receiving a responsive physiological signal. The detection light beam is incident into a fingertip of a user to form the responsive physiological signal. The wearable heart rate relay watch is connected with the detection finger band through a connection line. The wearable heart rate relay watch receives the responsive physiological signal and includes a gravitational accelerometer. The gravitational accelerometer senses the acceleration of the wearable heart rate relay watch to form an acceleration detection signal. A detection data includes the acceleration detection signal and the corresponding responsive physiological signal. The wearable heart rate relay watch outputs the detection data to an external device. An AI-based wearable heart-respiration monitoring system includes a control system communicating with the wearable respiration-heart monitoring kit.

### 1. FIELD OF THE INVENTION

The present invention relates to a physiological signal monitoring system, particularly to a physiological signal monitoring system for monitoring the heart condition, blood oxygen and respiration of a user in sleep at home.

### 2. DESCRIPTION OF THE PRIOR ART

Apnea is a sleep disorder, which the patient is unable to breathe and thus experiences interruption of respiration. The patient of apnea is often woken from sleep, and he can normally return to breathe after waking. In order to diagnose apnea, the patient needs to stay in a sleep center for one night clinically. In the sleep center, the patient will be examined with polysomnography (PSG), and the whole process is videoed. Items of polysomnography include Electroencephalography (EEG), Electrooculography (EOG), Electromyography (EMG), respiration air flow, blood pressure, respiratory effort, blood oxygen saturation (SpO2), Electrocardiogramaart rate, and sleeping posture, whereby to obtain the Apnea-Hypopnea Index (AHI). AHI is used to indicate the severity of sleeping respiration interruption, normally expressed by the number of respiration interruptions per hour, wherein a respiration interruption must be prolonged for at least 10 seconds and accompanied by decrease of blood oxygen saturation. In general, AHI 5-15 is a mild apnea, AHI 16-30 a moderate apnea, and AHI over 30 a severe apnea.

Sudden nocturnal deaths are 90% due to cardiovascular diseases, rarely due to severe viscus infections, pulmonary embolisms, or severe cerebrovascular accidents. In other words, most sudden deaths correlate with myocardial infarction, angina pectoris, cardiomyopathy, and arrhythmia. In some cases, arrhythmia may have occurred for several hours or one day before it is perceived clearly. However, there is no suitable instrument to detect arrhythmia and give an early waning.

Below are introduced some patents about apnea detection. A Taiwan patent No. 1754458 disclosed an instrument, a system and a method of detecting apnea, wherein three sensors respectively pick up snores, blood vessel parameters, and respiratory movements from the head, fingertip and trunk of the user and simultaneously cooperate with a clock to obtain a plurality of records of respiratory events; then the combinations of the respiratory events are used to detect apnea. A Taiwan patent No. 1711429 disclosed a wearable device, which is worn on the fingertip to measure health data of the user, such as the heart rate, blood oxygen saturation, and variation of heart rate. A Taiwan patent No. 11816104 disclosed an electronic device of detecting apnea and a method thereof, wherein a head-mounted apparatus transmits a wireless signal to the head of the testee and receives the reflected waves to obtain the detection results, whereby to determine whether the testee has apnea events. A Taiwan U.S. Pat. No. 1,728,839 disclosed a household sleep monitoring system, wherein an acoustic monitoring circuit is used to detect the snores of the testee; the respiratory parameters are obtained after analysis; the respiratory parameters are compared with the threshold values of respiratory events to obtain the classification of the respiratory events.

### SUMMARY OF THE INVENTION

A wearable kit of monitoring respiration and heart condition is provided, which is worn by the user in sleep to record and analyze the real-time physiological signals, and which further processes/analyzes the physiological signals with artificial intelligence, neither inconveniencing the user nor interfering with sleep.

A wearable kit of monitoring respiration and heart condition is provided, which includes a detection finger band and a wearable heart rate relay watch, which couple with each other in a wired way. The wearable heart rate relay watch has an accelerometer, able to exclude the posture changes during sleep, such as rolling over, getting up or picking up a call, with artificial intelligence, lest posture changes affect the record and analysis of physiological signals.

Accordingly, a wearable kit of monitoring respiration and heart condition is provided, which includes a detection finger band and a wearable heart rate relay watch. The detection finger band includes a detection light emitting unit, which emits a detection light beam, and a responsive light receiving unit, which receives a responsive physiological signal, wherein the detection light beam is incident into a fingertip to generate the responsive physiological signal. The wearable heart rate relay watch is connected with the detection finger band through a connection line. The wearable heart rate relay watch includes a gravitational accelerometer. The gravitational accelerometer detects the acceleration the wearable heart rate relay watch experiences to generate an acceleration detection signal. A piece of detection data includes the acceleration detection signal and the corresponding responsive physiological signals. The wearable heart rate relay watch transmits the detection data to an external device.

An artificial intelligence-based wearable kit of monitoring respiration and heart condition system, which has the abovementioned wearable kit of monitoring respiration and heart condition, further includes a control system communicating with the wearable heart rate relay watch. The control system uses an artificial-intelligence method to analyze the responsive physiological signals to obtain physiological information and then presents the responsive physiological signals and the physiological information.

The detection light beam includes a red light beam and an infrared light beam.

The wearable heart rate relay watch further includes a Type C connection port, which is connected with the connection line; and a transmission unit, which transmits the detection data to an external device in a near field wireless communication method.

The wearable heart rate relay watch further includes a processing unit, which processes the responsive physiological signals to obtain a plurality of physiological values. The physiological values include a respiration rate, a blood oxygen saturation rate, a pulse rate, and a perfusion index.

The external device includes a display device. The control system uses the display device to present the responsive physiological signals and the physiological information.

The control system includes an application program. The application program is installed in the external device and/or the wearable heart rate relay watch.

According to the acceleration detection signals detected within a period of time, the wearable heart rate relay watch determines whether to preserve or abandon the corresponding responsive physiological signals.

The wearable heart rate relay watch further includes a display unit. The display unit presents the responsive physiological signals and the physiological information.

### BRIEF DESCRIPTION OF THE DRAWINGS

FIG. 1 is a block diagram schematically showing a wearable kit of monitoring respiration and heart condition according to a first embodiment of the present invention.

FIG. 2 is a diagram schematically showing a scenario of using the wearable kit of monitoring respiration and heart condition according to the first embodiment of the present invention.

FIG. 3 is a diagram schematically showing a display interface of an application program collaborating with the wearable kit of monitoring respiration and heart condition according to the first embodiment of the present invention.

FIG. 4 is a diagram schematically showing a display interface of a cloud AI analysis function collaborating with the wearable kit of monitoring respiration and heart condition of the present invention.

### DESCRIPTION OF THE PREFERRED EMBODIMENT

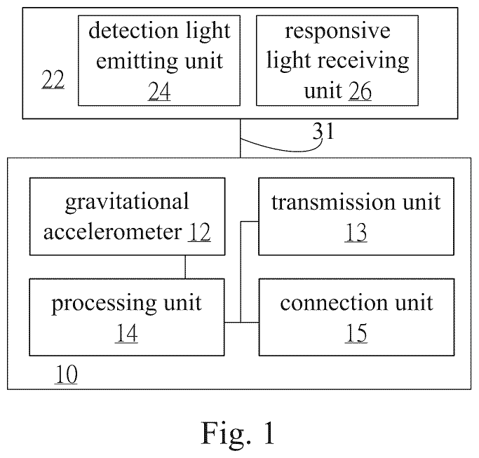

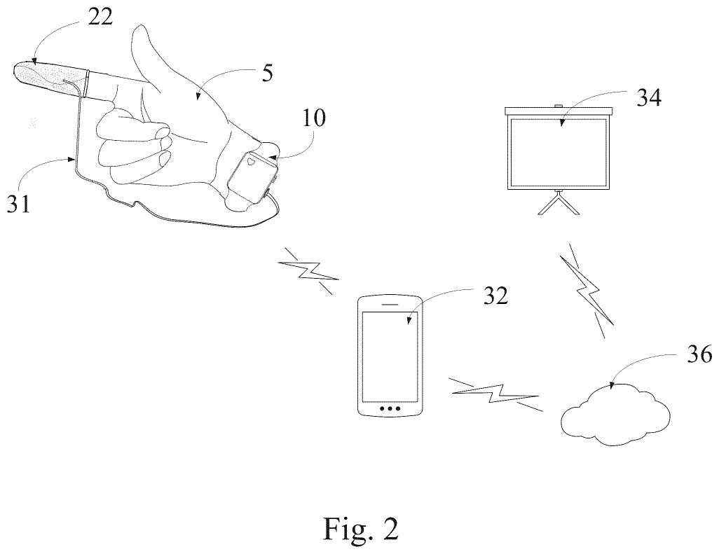

FIG. 1 is a block diagram schematically showing a wearable kit of monitoring respiration and heart condition according to a first embodiment of the present invention. FIG. 2 is a diagram schematically showing a scenario of using the wearable kit of monitoring respiration and heart condition according to the first embodiment of the present invention. Refer to FIG. 1 and FIG. 2. The wearable kit of monitoring respiration and heart condition of the present invention includes a detection finger band 22 and a wearable heart rate relay watch 10. The detection finger band 22 and the wearable heart rate relay watch 10 are separate and independent members. The detection finger band 22 is detachably connected with the wearable heart rate relay watch 10 through a connection line 31. In the first embodiment, the detection finger band 22 includes a detection light emitting unit 24 and a responsive light receiving unit 26. The wearable heart rate relay watch 10 includes a gravitational accelerometer 12, a processing unit 14, a transmission unit 13, and a connection unit 15. Each of the abovementioned units is formed by one or more modules/mechanisms/elements and disposed inside/on the casing of the wearable heart rate relay watch 10. It is easily understood: the wearable heart rate relay watch 10 may further include a power unit (not shown in the drawings) for suppling power to enable the operations of the abovementioned units. In practical application, the power unit may include primary batteries or secondary batteries. If the power unit includes secondary batteries, the power source unit may further include the circuit and mechanism required by secondary batteries.

Refer to FIG. 1 and FIG. 2 again. The detection finger band 22 has a physiological-signal interface, whereby the detection light emitting unit 24 may emit one or more detection light beams to react with a user 5. The detection light beam penetrates the skin of the user or is reflected by the skin of the user. Next, the responsive physiological signals (light signals) are received by the responsive light receiving unit 26 through the physiological-signal interface. Then, the responsive physiological signals are transmitted to the connection unit 15 of the wearable heart rate relay watch 10 through the connection line 31 for further processing. In one embodiment, the physiological-signal interface may be one or more openings exposed on the surface of the detection finger band 22. The detection light beam emitted by the detection light emitting unit 22 is incident into the detected position and reacts with the blood or tissue of the user 5 to form the responsive physiological signal, and the responsive physiological signal is received by the responsive light receiving unit 26. The detection light beam may be red light beam having a wavelength of 660 nm or infrared light beam having a wavelength of 940 nm. The responsive physiological signal includes blood oxygen and/or blood pressure pulse signals, heartbeat pulse signals, or perfusion index pulse signals, within a period of time, such as 10-30 seconds. The connection unit 15, such as a Type C connection port, may be connected with the connection line 31, and the connection line 31 can be plugged in or pulled out from the connection unit 15 easily. The responsive physiological signal coming from the detection finger band 22 is received by the connection unit 15 and then transmitted to the processing unit 14. The gravitational accelerometer 12 detects the accelerated states of the wearable heart rate relay watch 10 and persistently transmits the detection results to the processing unit 14. The processing unit 14 performs the pre-treatment of the responsive physiological signals and/or the detection results of the gravitational accelerometer 12, such as filtering, noise elimination, amplifying the signal-to-noise ratio, and analog-to-digital conversion. Then, the processing unit 14 transmits the pre-treated pulse signals and the gravitational acceleration detection results to the transmission unit 13 for sending out the signals.

Refer to FIG. 1 and FIG. 2 again. The transmission unit 13 of the wearable heart rate relay watch 10 transmits one or more pieces of the detection data to an external device 32 in a wireless near field communication technology, such as the Bluetooth technology, wherein each piece of detection data includes the real-time responsive physiological signal and the gravitational acceleration detection signal. The external device 32 may be equipped with an application program, which can collaborate with the wearable heart rate relay watch 10, to process and/or display one or more pieces of detection data. In one embodiment, the external device 32 may selectively transmit a portion of detection data to a cloud server 36 for further treatment in a wired or wireless method. Besides, the external device 32 may transmit the detection data to a display device 34 at a near end in a wired or wireless method to display the real-time responsive physiological signals. It is an option: the transmission unit 13 of the wearable heart rate relay watch 10 transmits one or more pieces of detection data to the display device 34 in a wireless near field communication method, such as the Bluetooth technology. The cloud server 36 includes application programs/software that use artificial intelligence to perform calculation/analysis, processing and analyzing the detection data transmitted to the cloud server 36 to obtain the information corresponding to blood pressure, respiration, blood oxygen, and heart status of the testee. It is an option: application programs/software using artificial intelligence to perform calculation/analysis may be installed in the wearable heart rate relay watch 10, the external device 32, or the display device 34.

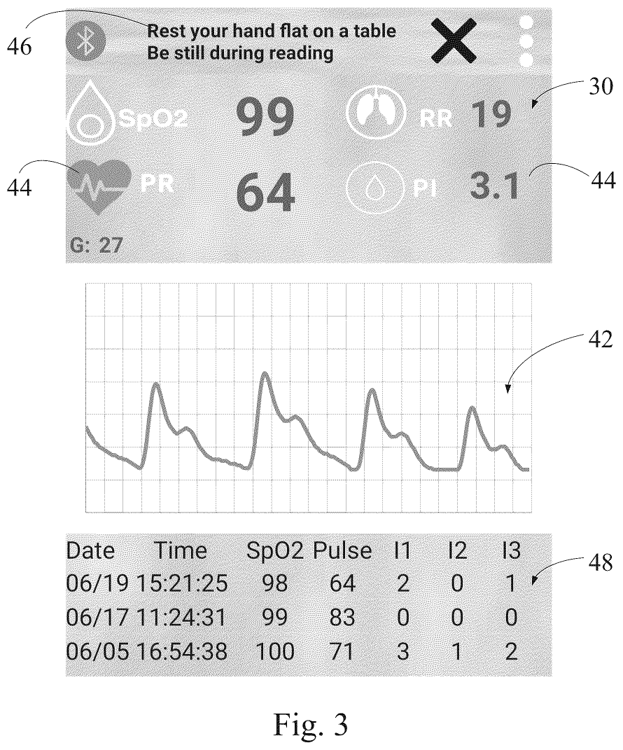

FIG. 3 is a diagram schematically showing a display interface of an application program collaborating with the wearable kit of monitoring respiration and heart condition according to the first embodiment of the present invention. Refer to FIGS. 1-3. Considering the scenario of using the wearable kit of monitoring respiration and heart condition, the wearable heart-respiration monitoring system of the present invention includes a wearable kit of monitoring respiration and heart condition and a control system. The control system is realized by an application program. The application program may be installed in the external device 32 (it may be a smart phone used nowadays), the display device 34 and/or the wearable heart rate relay watch 10 to display the measurement process of the wearable kit of monitoring respiration and heart condition. The user may use the external device 32 and/or the display device 34 and the corresponding program to start a display screen image 30. While the user uses the wearable heart rate relay watch 10 and the detection finger band 22 to perform measurement, the real-time pulses and the heartbeat pulses, which are measured by the wearable heart rate relay watch 10, may be transmitted to the external device 32 in a wireless method (such as the Bluetooth technology) and presented in the display screen image 30, including a plurality of physiological values 44 (such as the respiration rate (RR), the blood oxygen (SpO2), the pulse rate (PR), the perfusion index (PI)) and the heartbeat pulse graph 42. The display screen image 30 also may also present other indication information and records. For example, the display screen image 30 may also present one or more indication areas 46, including an indication area to remind the user of his posture in the measurement, an indication area to remind the user that the measurement is being undertaken, and an indication area to remind the user that the physiological signals are being uploaded to the cloud. However, the present invention is not limited by the abovementioned embodiment. It is an option: the wearable heart rate relay watch 10 may further include a display unit (not shown in the drawings). In such an embodiment, the display screen image 30 may be presented on the display unit of the wearable heart rate relay watch 10.

Refer to FIGS. 1-3 again. For the patients suffering from apnea, wearing a medical ventilator in sleep is an unavoidable choice. Occurrence of apnea is likely to be accompanied by a series of body reactions, such as abnormal heartbeat. Therefore, it is very important for apnea patients to monitor the physiological states in sleep, such as the states of respiration and heartbeat. However, conscious or unconscious movements in sleep, such as rolling over and lifting up/laying down the limbs, may make the monitoring instruments unable to detect accurate physiological signals and make the succeeding processing/analysis output incorrect results. Hence, the wearable heart rate relay watch of the present invention is equipped with a gravitational accelerometer to detect acceleration in real time, and the acceleration signals together with the responsive physiological signals form the detection data. Whether the responsive physiological signals detected within a period of tome are to be preserved or abandoned according to the acceleration signals detected within the period of time, whereby to increase the correctness of the responsive physiological signals.

Refer to FIGS. 1-3 again. The wearable kit of monitoring respiration and heart condition of the present invention enables the user to simultaneously record and monitor the respiration and heart status in sleep easily and conveniently. While the user wears the wearable kit of monitoring respiration and heart condition of the present invention in sleep, the kit can detect the accelerations of the user and the responsive physiological signals in real time in sleep. The wearable heart rate relay watch 10 can detect the accelerations occurring during a series of or a single cycle of rolling over, turning over, or lifting up/laying down a limb in sleep. Whether the responsive physiological signals within a period of time are to be abandoned or not is determined via analyzing the acceleration variations occurring during the period of time.

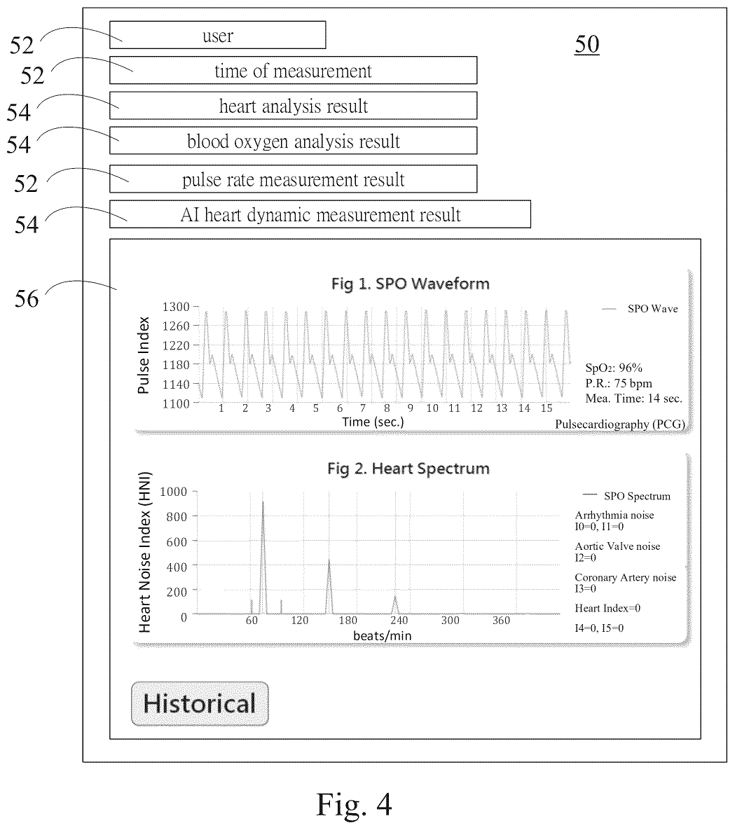

FIG. 4 is a diagram schematically showing a display interface of a cloud AI analysis function collaborating with the wearable kit of monitoring respiration and heart condition of the present invention. Refer to FIGS. 1-4 simultaneously. The external device 32 receives the measurement data from the wearable heart rate relay watch 10 and presents the measurement data on the display screen image 30. At the same time, the external device 32 transmits one or more pieces of measured acceleration data and corresponding responsive physiological signals to the cloud server of the network for analysis in a wired method or a wireless method (such as the Bluetooth technology). In one embodiment, the cloud server includes the application programs/software that uses artificial intelligence to perform calculation/analysis, able to process and analyze the acceleration data and corresponding responsive physiological signals, which are transmitted to the cloud server, with artificial intelligence to obtain the information corresponding to respiration rate, pressure oxygen, pulse rate, and heart status of the testee. For example, after the cloud server analyzes the responsive physiological signals with artificial intelligence and obtains the results, the external device 32 and/or the display device 34 may access the Internet to look up the blood oxygen/heart status monitoring list 50. The blood oxygen/heart status monitoring list 50 may include but is not limited to include one or more basic/measurement data display areas 52, one or more analysis result display areas 54, and one or more measurement/analysis graph display areas 56. The information presented by basic/measurement data display areas 52 includes the user's name and the user's code. The measurement data includes the measurement date and the measurement results. The information displayed by the measurement/analysis graph display areas 56 includes the blood oxygen waveform graph and the heart spectrum waveform graph. The analysis result display areas 54 display the physiological information obtained via analyzing the responsive physiological signals. For example, the analysis result display areas 54 display the heart status is normal, the blood oxygen is normal, and the pulse rate is normal. It is an option: the physiological information displayed by the analysis result display areas 54 may also be presented in the analysis result display areas 48 of the display screen image 30. It is an option: the application programs/software that uses artificial intelligence may also be installed in the wearable heart rate relay watch 10.

The embodiments described above are to demonstrate the technical thoughts and characteristics of the present invention to enable the persons skilled in the art to understand, make, and use the present invention. However, these embodiments are not intended to limit the scope of the present invention. Any equivalent modification or variation according to the spirit of the present invention is to be also included by the scope of the present invention.

What is claimed is:

1. A wearable kit of monitoring respiration and heart condition, comprising
a detection finger band, including a detection light emitting unit and a responsive light receiving unit, wherein the detection light emitting unit emits a detection light beam; the responsive light receiving unit receives a responsive physiological signal, and wherein the detection light beam is incident into a fingertip of a user to generate the responsive physiological signal; and
a wearable heart rate relay watch, connected with the detection finger band through a connection line, receiving the responsive physiological signal through the connection line, including a gravitational accelerometer, wherein the gravitational accelerometer detects acceleration of the wearable heart rate relay watch to generate an acceleration detection signal; detection data includes the acceleration detection signal and the corresponding physiological signal; and the wearable heart rate relay watch transmits the detection data to an external device.

2. The wearable kit of monitoring respiration and heart condition according to claim 1, wherein the detection light beam includes a red light beam and an infrared light beam.

3. The wearable kit of monitoring respiration and heart condition according to claim 1, wherein the wearable heart rate relay watch includes a Type C connection port which is connected with the connection line; and a transmission unit which transmits the detection data to the external device in a near-field wireless communication method.

4. The wearable kit of monitoring respiration and heart condition according to claim 1, wherein the wearable heart rate relay watch further includes a processing unit which processes the responsive physiological signals to obtain a plurality of physiological values, wherein the physiological values include a respiration rate, a blood oxygen saturation level, a pulse rate, and a perfusion index.

5. The wearable kit of monitoring respiration and heart condition according to claim 4, wherein the wearable heart rate relay watch further includes a display unit; the processing unit processes and analyzes the responsive physiological signals with artificial intelligence to obtain the physiological values; the display unit presents the responsive physiological signals and the physiological values.

6. An artificial intelligence-based wearable kit of monitoring respiration and heart condition system with the wearable kit of monitoring respiration and heart condition according to claim 1, further comprising a control system which communicates with the wearable heart rate relay watch, wherein the control system analyzes the responsive physiological signals with artificial intelligence to obtain physiological information and present the responsive physiological signals and the physiological information.

7. The artificial intelligence-based wearable kit of monitoring respiration and heart condition system according to claim 6, wherein the external device includes a display device; the control system uses the display device to present the responsive physiological signals and the physiological information.

8. The artificial intelligence-based wearable kit of monitoring respiration and heart condition system according to claim 6, wherein the control system includes an application program; the application program is installed in the external device and/or the wearable heart rate relay watch.

9. The artificial intelligence-based wearable kit of monitoring respiration and heart condition system according to claim 6, wherein the wearable heart rate relay watch determines whether to preserve or delete corresponding physiological signals according to the acceleration detection signals detected within a period of time.

10. The artificial intelligence-based wearable kit of monitoring respiration and heart condition system according to claim 6, wherein the wearable heart rate relay watch further includes a display unit which presents the responsive physiological signals and the physiological information.
